(19) United States
(12) Patent Application Publication
(10) Pub. No.: US 2010/0292601 A1
(43) Pub. Date: Nov. 18, 2010

(54) APPARATUS FOR AND METHOD OF CONDENSING EXHALED BREATH

(75) Inventors: Edward Dompeling, Berg En Terblijt (NL); Quirijn Jobsis, Bemelen (NL); Charlotte Magdalena Hubertus Robroeks, Maastricht (NL); Franciscus Johannes Theresia Nijpels, Maastricht (NL); Johannes Eugene Aerts, Oost-Maarland (NL)

Correspondence Address: FISH & RICHARDSON P.C., P.O BOX 1022, Minneapolis, MN, 55440-1022, US

(73) Assignee: UNIVERSITEIT MAASTRICHT, Maastricht (NL)

(21) Appl. No.: 12/527,025

(22) PCT Filed: Feb. 15, 2008
(86) PCT No.: PCT/EP08/51849

§ 371 Date: May 24, 2010

(30) Foreign Application Priority Data
Feb. 16, 2007 (EP) 07102586.0

(51) Int. Cl. A61B 5/097

(52) U.S. Cl. 600/543

(57) ABSTRACT

A breath condensing apparatus includes a mouthpiece, a condenser having an inlet coupled to the mouthpiece, a first container for collecting condensate communicating with an outlet of the condenser, and a second container for collecting residual, non-condensed exhaled breath coupled to the condenser. A method of condensing exhaled breath includes the features of receiving exhaled vapor into a condenser, condensing at least part of the exhaled vapor, collecting at least part of residual, non-condensed exhaled vapor, recirculating at least part of the collected residual vapor through the condenser, condensing at least part of the recirculated vapor, and collecting condensate.

### RELATED APPLICATIONS

This application is a 35 U.S.C. §371 national phase of International Application No. PCT/EP2008/051849, entitled “APPARATUS FOR AND METHOD OF CONDENSING EXHALED BREATH” filed on Feb. 15, 2008, which claims priority to European Application Serial Number 07102586.0, filed on Feb. 16, 2007.

### TECHNICAL BACKGROUND

The present disclosure relates to an apparatus and method for condensing exhaled breath.

### BACKGROUND

Various apparatus and techniques have been utilized for condensing exhaled breath. For instance, international patent application WO 00/35337 relates to a device and method for non-invasively monitoring asthma and other respiratory diseases. The method includes collecting condensate from a subject's breath, testing the condensate to determine its acidity level or ammonium concentration, and evaluating these properties to determine the presence, absence or status of a respiratory disease in the subject.

The embodiment shown in FIG. 1 of WO 00/35337 includes a mouthpiece apparatus (20), a condensation apparatus (60) having a through passage (69), and a collection apparatus (100). Mouthpiece apparatus 20 has a proximal end 22 and a distal end 24, and includes a mouthpiece 26 configured to sealingly engage one or more of a subject's mouth, nose or artificial airway. “Optionally, after a few drops of condensate have been obtained, the circuit formed by condensation apparatus 60 and collection apparatus 100 may be closed and means may be provided for recirculating exhalate through it in order to propel additional condensate through passage 69 and into collection chamber 102.” (page 19, line 18 to page 20, line 12).

For the sake of completeness, attention is drawn to P. R. R. Rosias, et al., “Exhaled breath condensate in children: Pearls and pitfalls”, Pediatric Allergy and Immunology, 2004:15, pages 4-19, and to P. R. R. Rosias, et al., “Breath condenser coatings affect measurement of biomarkers in exhaled breath condensate”, European Respiratory Journal, 2006, Volume 28, number 5.

WO 95/31721 relates to a process and a device for collecting expired breath condensate for the diagnosis of the state of health and metabolic efficiency of the lung and respiratory tracts and other organs and to analyze expired substances foreign to the body, in which the expired air is cooled and the non-gaseous components are condensed out in a test vessel. In WO 95/31721, the expired air passes through a sample collecting tube and is cooled therein to a negative temperature below 0° C., whereby the liquid and soluble components condense out and freeze on the inner wall of the sample collecting tube.

WO 02/082997 relates to a control device for setting a breathing gas pressure in the field of diagnosis and/or therapy of sleep-related respiratory disorders.

US 2003/0208133 relates to a respiratory analyzer for a person comprising a flow path through which the person breathes; a metabolic rate meter, providing a metabolic rate for the person; a ketone sensor, providing a ketone signal related to the concentration of respiratory components correlated with a level of ketone bodies in exhalations of the person; a display; and an electronic circuit, receiving the ketone signal and the metabolic data, and providing a visual indication of the metabolic rate and the ketone signal to the person on the display.

US 2005/0137491 relates to aerosol traps and to a method and apparatus for collecting aerosols in exhaled breath for diagnostic and other purposes. The apparatus comprises an aerosol collection chamber with a collection surface for collecting aerosol particles and/or droplets from exhaled breath; a conduit for channeling the exhaled breath from the test subject to the aerosol collection chamber; and a pre-collection filter positioned in close enough proximity to the aerosol collection chamber to accommodate filtering ambient air inhaled by the test subject that is comprised in said exhaled breath of the test subject.

US 2004/138577 relates to a breath condensate collection apparatus comprising a central chamber, a breath input assembly, a plunger assembly and a breath condensate collection port.

EP 577 053 discloses a gas collecting tube which can mount at one end an expired gas collecting mask and has at the other end a bag made of thin plastic and having an aperture at the end so that the bag is swollen by patients' breathing out while a part of expired gas leaks through the aperture to allow the breathing-out by patients to be continued, whereby patients blowing-out expired gas can be checked by watching swelling of the bag.

### SUMMARY

The invention relates to an apparatus for condensing exhaled breath, including a mouthpiece, a condenser having an inlet communicating with the mouthpiece, and a container for collecting condensate communicating with an outlet of the condenser. The invention also relates to a method of condensing exhaled breath.

It is one object of the present invention to more efficiently condense exhaled breath, thus being able to reduce the time necessary to obtain a sample, which is significant for e.g., minimally cooperative patients (typically infants less than four years old), and/or to obtain more condensate and/or higher concentrations in the condensate of e.g., markers of airway inflammation.

To this end, one embodiment of the apparatus according to the present disclosure is characterized by a further container for collecting residual, non-condensed exhaled breath, which container communicates with the condenser.

In some aspects, it is preferable that the further container is a collapsible bag made of a flexible, thin-walled material, preferably a film of an inert, impermeable synthetic material, such as Tedlar™ or a polyolefin, such as polyethylene (PE). Suitable wall thicknesses include, but are not limited to, those in a range from 10 to 250 μm, more specifically from 15 to 100 μm.

In some aspects, it is further preferable that the container for residual breath is connected to both the inlet and the outlet of the condenser, thus facilitating recirculation of breath collected in this container through the condenser.

In a further aspect of the present disclosure, the container for residual breath has a volume of more than 10 liters, preferably more than 20 liters or more preferably more than 30 liters. Typically, a volume of more than 100 liters is not required, although certain applications, such as condensing breath of larger animals, for instance horses, may require larger volumes.

In some aspects, the apparatus for condensing exhaled breath may include one or more heating elements for maintaining residual, non-condensed exhaled breath gaseous and preferably at a temperature of at least 30° C., more preferably in a range from 35° C. to 42° C., thus ensuring that, on the one hand, most of the residual breath will indeed remain gaseous and, on the other hand, preventing substances, in particular markers, contained in the residual breath from deteriorating.

In another embodiment of the invention, an apparatus for condensing exhaled breath includes a mouthpiece, a condenser having an inlet in fluid communication with the mouthpiece, a container for collecting condensate in fluid communication with an outlet of the condenser, and one or more heating elements for maintaining residual, non-condensed exhaled breath gaseous and at a temperature of between approximately 30° C. and approximately 42° C.

Various embodiments of the invention also relate to a method of condensing exhaled breath including the steps of: having a subject exhale into a condenser, condensing part of the exhaled breath, collecting at least part of residual, non-condensed exhaled breath, recirculating at least part of the collected residual breath through the condenser or a further condenser, condensing at least part of the recirculated breath, and collecting condensate.

In some aspects, it is preferable that residual, non-condensed air is collected in a collapsed bag.

In another embodiment of the invention, a method for condensing exhaled breath includes the features of receiving exhaled breath into a condenser, condensing at least part of the exhaled breath, heating the residual, non-condensed exhaled breath to a temperature of between approximately 30° C. and 42° C., recirculating at least part of the heated residual breath through the condenser, condensing at least part of the recirculated breath, and collecting condensate.

### DESCRIPTION OF DRAWINGS

The invention will now be explained in more detail with reference to the figures, which show a presently preferred embodiment of the apparatus according to the present disclosure.

FIG. 1 is cross-section of an apparatus for condensing exhaled breath.

FIGS. 2, 3, 5A-7 are detailed cross-sections of, respectively, a mouthpiece, condenser in- and outlets, and container in- and outlets of the apparatus shown in FIG. 1.

FIG. 4 is a perspective view of a wiper shown in FIG. 3.

It is noted that the drawings are not necessarily to scale and that details, which are not required for understanding the present invention, may have been omitted. The terms “upward,” “downward,” “top,” “bottom,” and the like relate to the embodiments as oriented in the Figures.

### DETAILED DESCRIPTION

FIG. 1 shows an apparatus 1 for condensing exhaled breath (EBC), including a mouthpiece unit 2, an inclined, double-walled condenser tube 3 having, at or near its top, an inlet 4A coupled to the mouthpiece unit 2, and a container 5 for collecting condensate communicating with the central lumen of the condenser tube 3.

The apparatus 1 further includes a gas container 6 for collecting residual breath, as will be explained in detail below, housed in a cabinet 7 and communicating with the outlet 4B of the condenser tube 3 via a connecting tube 8. The gas container 6 also communicates (e.g., in fluid communication) with the inlet 4A of the condenser 3, in this example via a cut-off valve 9 and a further connecting tube 10.

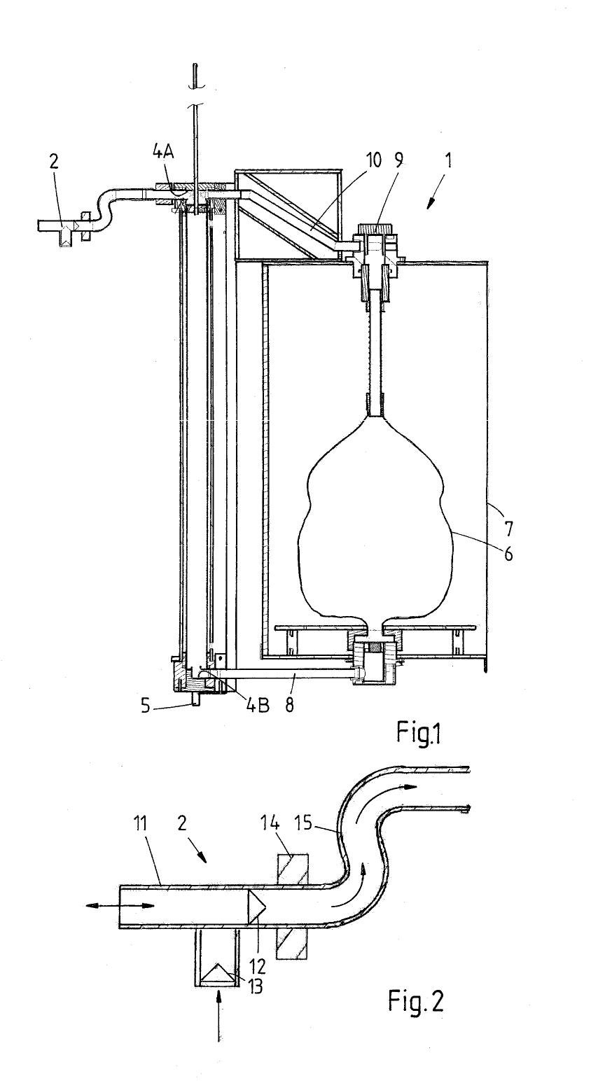

The mouthpiece unit 2, shown in detail in FIG. 2, includes the actual mouthpiece 11 for a subject to blow in, a first non-return valve 12, which prevents backflow when the subject inhales, and a second non-return valve 13, located in a wall of the mouthpiece 11 and upstream from the first valve 12, thus allowing the patient to inhale without having to remove the mouthpiece 11. The unit 2 further includes a flow meter, in this example a Doppler flow meter 14, and a saliva-trap, in this example a swan-neck 15. The mouthpiece 11 can be attached to or form an integral whole with a mask (not shown) to be attached to the subject's face. Such a facemask may include an internal baffle to isolate breath coming from the mouth from breath coming from the nose, thus avoiding cross-contamination.

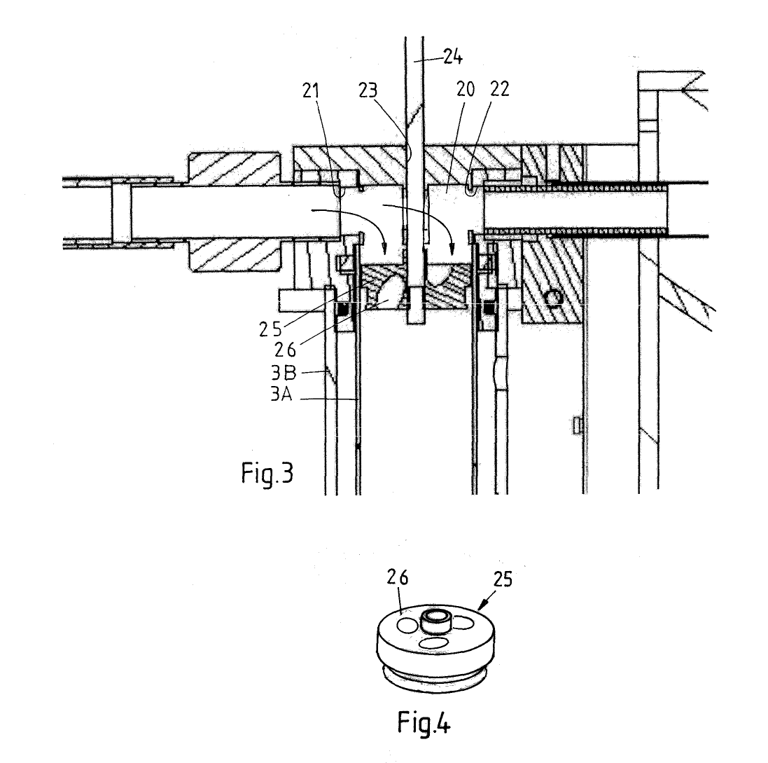

The inlet 4A, shown in detail in FIG. 3, of the condenser tube 3 includes a central chamber 20 having two radial openings 21, 22, connected to the mouthpiece unit 2 and to the air container 6, respectively, and an axial channel 23, slidingly accommodating a rod 24. In this example, the rod 24 is longer than the condenser tube 3 and is provided on its lower end with a wiper, e.g., a plunger 25 sealingly abutting the inner wall of the cylindrical inner tube 3A of the condenser 3. The plunger 25, shown in more detail in FIG. 4, includes a plurality of channels 26 located downstream from the openings 21, 22, and having both a tangential and an axial component, e.g., extending at about 60° relative to the imaginary central axis of the condenser tube 3.

The cylindrical inner and outer tubes 3A, 3B of the double-walled condenser tube 3 together define an annular lumen, which is connected, at or near the bottom and the top of the condenser tube 3, to a cooling device (not shown). Although the inner wall of the inner tube 3A can be provided with a coating, such as Teflon™, silicone or aluminum, in this example the inner tube 3A is made of non-coated glass. The outer tube 3B is made of glass or a clear synthetic material, such as polycarbonate. Further, the condenser tube preferably has a length of at least 50 cm, in this example 90 cm.

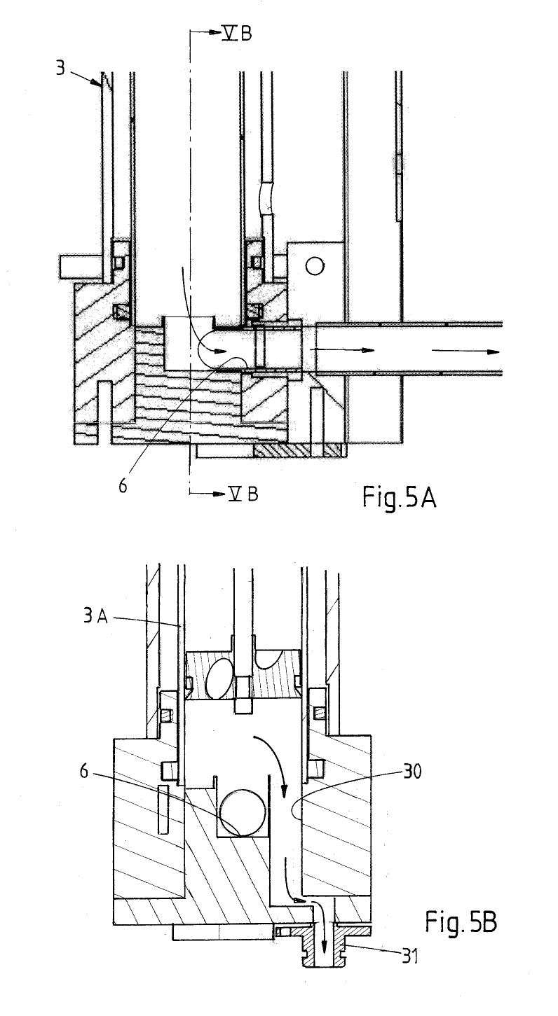

FIGS. 5A and 5B show cross-sectional side and front views, respectively, of the bottom section of the condenser tube 3, which section includes a liquid trap 30 in line with the right-hand side (in FIG. 5B) of the inner tube 3A so as to effectively collect condensate from the inner wall of the inner tube 3A under the influence of gravity and to prevent condensate from flowing into the outlet 4B. The bottom section of the condenser 3 further includes a connector 31 for releasably attaching the container 5 for collecting condensate (as shown in FIG. 1). Optionally, the container can be provided with one or more features, such as a double wall similar to the condenser tube, for cooling the collected condensate and/or with one or more features, e.g., a so-called lab on a chip, for carrying out a single or multiple lab processes, e.g., detecting one or more specific markers, such as H2O2 and 8-isoprostane, for (preliminary) diagnosis.

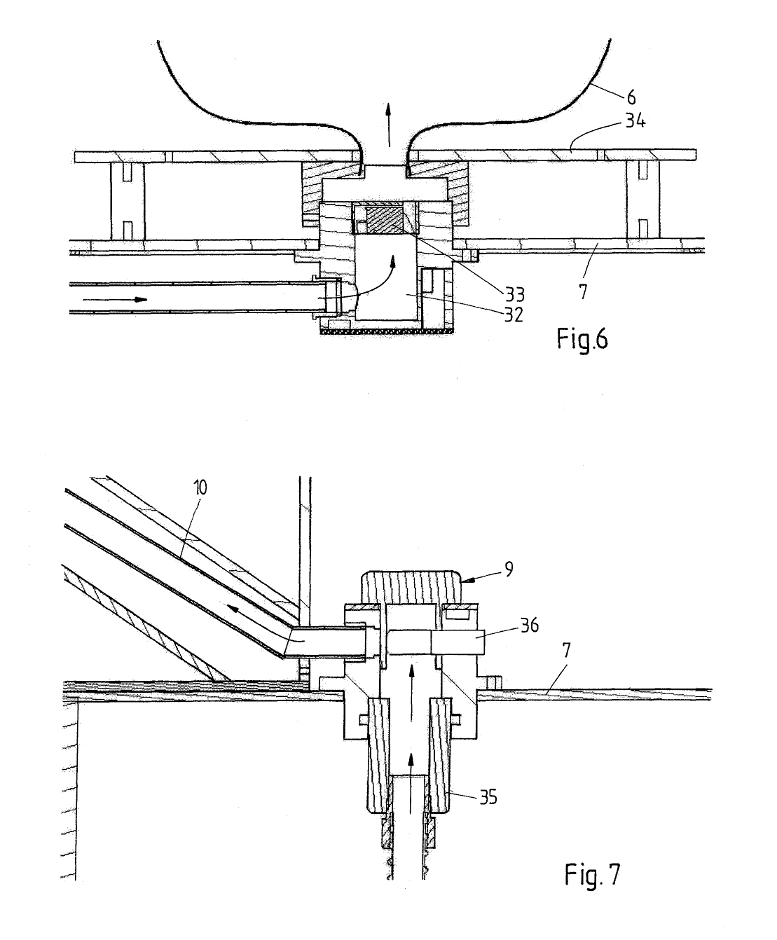

As depicted in FIG. 6, the lower section of the cabinet 7 includes a chamber 32, accommodating a ventilator 33 for forced recirculation of breath, as will be explained below. One or more heating elements 34, coupled to suitable control features (not shown), are mounted inside the cabinet 7 for maintaining a set temperature, e.g. in a range from 35 to 37° C. FIG. 6 further shows the lower part of the gas container 6, which, in this example, is a bag made of a flexible film of an inert synthetic material, such as a fluoropolymer, e.g., Tedlar™, or a polyolefin, e.g., polyethylene (PE), and having a wall thickness in a range from 10 to 50 μm, e.g., 25 μm.

FIG. 7 shows details of the cut-off valve 9 connecting a mount 35 for the gas container 6 to the inlet 4A of the condenser tube 3. A humidity sensor 36 is located in a sidewall of the valve 9.

According to some example embodiments, the apparatus 1 is operated as follows. First, a disposable, sterile gas container 6 is placed, in collapsed, substantially empty condition, in the cabinet 7 and a sterile condenser tube 3 is attached to one of the sidewalls of the cabinet 7. The container 6 and condenser tube 3 are interconnected to enable recirculation of breath collected in the gas container 6 through the condenser tube 3. The cut-off valve 9 is closed and the plunger 25 is pulled to its uppermost position.

A subject exhales into the mouthpiece 11, causing the first non-return valve 12 to open and allowing breath to enter the inlet 4 of the condenser tube 3 via the swan-neck 15, which serves a saliva-trap and thus prevents contamination of the exhaled breath. Exhaled breath subsequently flows through the inclined channels 26 in the plunger 25 and is thus directed turbulently towards the inner wall of the inner tube 3A, similar to a cyclone, to enhance condensation. Data from the flow meter 14 can be used to calculate the amount of breath exhaled and hence the amount of breath collected in the gas container 6.

When the subject inhales, the first non-return valve 12 closes and the second valve 13 opens to allow surrounding air to enter.

A coolant, e.g., water at or substantially at 0° C., flows through the annular (outer) lumen of the condenser tube 3 in countercurrent (from bottom to top in the Figures), thus cooling breath entering the condenser and flowing downwards and causing non-volatile components of the exhaled breath to condensate on the inner wall of the condenser tube 3. It is generally preferred that the condensate remains a liquid, i.e., does not freeze.

Non-condensed exhaled breath leaves the condenser 3 through the bottom outlet 4B and, via the connecting tube 8, enters the gas container 6, which is thus gradually inflated. The temperature inside the cabinet 7 and hence inside the container 6 is maintained at about 37° C. so as to ensure that, on the one hand, collected breath will remain gaseous and, on the other hand, the substances contained in the collected breath will not deteriorate.

When a sufficient amount of breath has been collected, the plunger 25 is pushed downwards so as to wipe any condensate from the inner wall of the condenser tube 3 and towards the container 5 for collecting condensate.

Subsequently, the plunger 25 is pulled up again, the cut-off valve 9 is opened, and the ventilator 33 is activated, i.e., non-condensed residual breath in the gas container 6 is recirculated through the condenser 3, preferably from top to bottom to assist a downward flow of condensate, i.e., towards the container 5.

Recirculation is stopped when the relative humidity drops to approximately 10% or less. Subsequently, the plunger 5 is subsequently pushed downwards once more to wipe condensate from the inner wall of the condenser tube 3.

With the apparatus and method according to the present disclosure, more condensate can be produced from the same or a smaller volume of breath. This increase in efficiency can be employed to reduce the time necessary to obtain a sample, e.g., to reduce the time a patient must be exposed to testing. Alternatively or additionally, the increase in efficiency can be employed to obtain higher amounts or, for some substances, even higher concentrations of e.g., markers of (airway) inflammations or other conditions. Hence, the apparatus and method according to the present disclosure enables feasible collection of breath condensate in e.g., children aged zero to four years, a more rapid collection in older children or adults, and a higher and more reproducible yield of EBC, in turn enabling a more accurate diagnosis of airway inflammations, other (non-airway) inflammations, and possibly even other conditions, such as infections and metabolic or cardiac disorders.

Another embodiment of the present disclosure, not shown in the Figures, includes two or more parallel condensers and a valve for distributing exhaled breath from the mouthpiece over these condensers. For instance, breath exhaled first typically originates from the larger bronchial parts of the lungs, whereas breath exhaled last typically originates more from the distal or even alveolar parts. By switching, during one exhalation, from one condenser to the next breath and thus markers indicative of inflammation of different parts of the lungs can be separated effectively, enabling a more accurate and specific diagnosis or a comparison between inflammation in bronchial and alveolar parts of the lungs.

The invention is not restricted to the above-described embodiments, which can be varied in a number of ways within the scope of the claims. For instance, the apparatus can be coupled to another apparatus for drying the inhaled air, preferably to 10% relative humidity or less, to extract more water/moist from the subject during respiration. Also, one or more components of the apparatus according to the present disclosure, in particular the collapsible container and/or the condenser tube, can be disposable to reduce the risk of contamination between samples.

In yet another example, the condenser includes two or more inner tubes so as to increase the surface area available for condensation. In such an embodiment, it is preferred that each of the inner tubes is provided with a plunger. Also, if two or more tubes are used the length of the condenser can be relatively small, e.g., smaller than 50 cm, e.g., 30 cm, which renders it more suitable for use by general practitioners.

1-15. (canceled)

16. A breath condensing apparatus, the apparatus comprising:
a mouthpiece,
a condenser having an inlet in fluid communication with the mouthpiece,
a first container for collecting condensate in fluid communication with an outlet of the condenser, and
a second container for collecting residual, non-condensed exhaled breath in fluid communication with the condenser.

17. The apparatus according to claim 16, wherein the second container is a collapsible bag made of a flexible, thin-walled material.

18. The apparatus according to claim 17, wherein the flexible, thin-walled material comprises a film of an inert synthetic material.

19. The apparatus according to claim 16, wherein the second container is connected to both the inlet and the outlet of the condenser thus enabling recirculation of breath collected in the second container through the condenser.

20. The apparatus according to claim 16, wherein the second container has a volume of between approximately 10 liters and approximately 100 liters.

21. The apparatus according to claim 16, further comprising one or more heating elements for maintaining residual, non-condensed exhaled breath gaseous and at a temperature of between approximately 30° C. and approximately 42° C.

22. The apparatus according to claim 21, further comprising a casing, wherein the second container and the heating elements are disposed inside the casing.

23. The apparatus according to claim 16, further comprising a wiper positionable inside the condenser to wipe condensate from the inner wall of the condenser and towards the first container for collecting condensate.

24. The apparatus according to claim 16, further comprising one or more channels located at or below the inlet and having both a tangential and an axial component, relative to the central axis of the condenser, to direct exhaled breath towards the inner wall of the tube.

25. The apparatus according to claim 16, wherein at least one of the mouthpiece or a duct connecting the mouthpiece to the condenser comprises one of:
a two-way non-rebreathing valve system,
a flow meter, and
a saliva-trap.

26. The apparatus according to claim 16, further comprising a sensor for measuring the relative humidity of the contents of the second container.

27. The apparatus according to claim 16, further comprising a ventilator adapted to force recirculation of non-condensed exhaled breath.

28. A method of condensing exhaled breath, the method comprising:
receiving exhaled breath from a subject into a condenser,
condensing at least part of the exhaled breath,
collecting at least part of residual, non-condensed exhaled breath,
recirculating at least part of the collected residual breath through the condenser,
condensing at least part of the recirculated breath, and
collecting condensate.

29. The method according to claim 28, further comprising collecting the residual, non-condensed air in a collapsible bag.

30. The method according to claim 29, wherein the bag is collapsed when exhaled breath is received into the condenser.

31. The method according to claim 28, further comprising heating the residual, non-condensed exhaled breath to a temperature of between approximately 30° C. and 42° C.

32. The method of claim 28, wherein condensing at least part of the exhaled breath comprises condensing at least 10 liters of the exhaled breath.

33. The method of claim 28, the condenser comprising a first condenser and a second condenser, wherein receiving exhaled breath into a condenser comprises receiving exhaled breath into the first condenser, and recirculating at least part of the collected residual breath through the condenser comprises recirculating at least part of the collected residual breath through the second condenser.

34. A breath condensing apparatus, the apparatus comprising:
a mouthpiece,
a condenser having an inlet in fluid communication with the mouthpiece,
a container for collecting condensate in fluid communication with an outlet of the condenser, and
one or more heating elements for maintaining residual, non-condensed exhaled breath gaseous and at a temperature of between approximately 30° C. and approximately 42° C.

35. The apparatus according to claim 34, further comprising a casing, wherein the heating elements are disposed inside the casing.

36. A method of condensing exhaled breath, the method comprising:
receiving exhaled breath into a condenser,
condensing at least part of the exhaled breath,
heating the residual, non-condensed exhaled breath to a temperature of between approximately 30° C. and 42° C.,
recirculating at least part of the heated residual breath through the condenser,
condensing at least part of the recirculated breath, and
collecting condensate.

37. The method of claim 36, the condenser comprising a first condenser and a second condenser, wherein receiving exhaled breath into a condenser comprises receiving exhaled breath into the first condenser, and recirculating at least part of the heated residual breath through the condenser comprises recirculating at least part of the heated residual breath through the second condenser.
